(19) United States
(12) Patent Application Publication
(10) Pub. No.: US 2010/0105867 A1
(43) Pub. Date: Apr. 29, 2010

(54) PROCESS FOR PRODUCING OSTEOCALCIN-CONTAINING EXTRACT

(75) Inventors: Hiromu Ohnogi, Shiga (JP); Shinichi Adachi, Shiga (JP); Kazuo Nakagawa, Shiga (JP); Hiroki Iwamoto, Shiga (JP); Ikunoshin Kato, Shiga (JP)

Correspondence Address: BROWDY AND NEIMARK, P.L.L.C.;624 NINTH STREET, NW, SUITE 300, WASHINGTON, DC, 20001-5303, US

(73) Assignee: TAKARA BIO INC., SHIGA (JP)

(21) Appl. No.: 12/530,270

(22) PCT Filed: Mar. 5, 2008
(86) PCT No.: PCT/JP2008/053927

§ 371 Date: Sep. 16, 2009

(30) Foreign Application Priority Data
Mar. 6, 2007 (JP) 2007-056420
Jun. 25, 2007 (JP) 2007-166627
Aug. 10, 2007 (JP) 2007-210391

(51) Int. Cl. A61K 38/16; A61P 1/02; C07K 14/00

(52) U.S. Cl. 530/350

(57) ABSTRACT

A process for producing an extract containing activated osteocalcin; an extract which can be obtained by this production process; a food, a drink, a drug, an oral cavity-care composition and a feed containing this extract; and a growth enhancer containing osteocalcin originating in a bone.

### TECHNICAL FIELD

The present invention relates to a process for producing an extract containing active osteocalcin; an extract obtainable by the production process; a food or beverage product, a medicament, an oral care composition and a feed that contain the extract; as well as a growth enhancer containing osteocalcin obtained from a bone.

### BACKGROUND ART

Active osteocalcin, also called as bone Gla-containing protein, is a characteristic non-collagenous protein that is synthesized by osteoblasts, bone forming cells, and that is present in extracellular matrix. Active osteocalcin reportedly accounts for 10 to 20% of bone non-collagenous proteins.

Human osteocalcin contains 49 amino acids in the molecule, in which glutamic acid residues in the molecule are converted to γ-carboxyglutamic acid (Gla) residues by vitamin K-dependent post-translational modification. γ-Carboxylated osteocalcin contains three residues of Gla (at 17, 21 and 24 positions) in the molecule. The biosynthesis of γ-carboxylated osteocalcin in osteoblasts is enhanced by active vitamin D. γ-Carboxylated osteocalcin is called active osteocalcin since it is capable of binding to calcium via the Gla residues and is reportedly involved in calcification of bones because of this function. Serum osteocalcin levels are used as an index of bone formation and metabolic bone diseases because a part of the osteocalcin molecule is secreted into blood.

In human beings, lower intake of minerals than the requirement is undesirable for maintaining good health. In particular, lack of calcium is known not only to increase the risk of bone diseases such as osteoporosis, but also to affect the functions of muscles and nervous tissue. In order to enhance the absorption of minerals, including calcium, utilization of Gla, which has an affinity for calcium, as a mineral absorption enhancer has been studied; and Gla-containing osteocalcin reportedly may be used as a material for such an enhancer (Patent Document 1).

There are only a few reports of processes for producing active osteocalcin from biological materials. A method for selectively solubilizing osteocalcin and osteopontin from a chicken bone has been reported (Non-Patent Document 1). The method disclosed in this document is extraction with a solvent containing ethylenediaminetetraacetic acid (EDTA).

Besides the above-mentioned extraction method using a solvent containing EDTA, an extraction method with an acid is known for extracting osteocalcin from a bone; however, the extraction efficiency of these methods is unsatisfactory (Non-Patent Document 2).

On the other hand, bones of livestock animals are extracted with hot water, and the extracts are used as raw materials for food soup and natural seasonings. However, bone residues after extraction have no effective use; at most used as fertilizers if they are reused. Thus, more effective applications of bone residues have been desired.

Patent Document 1: JP 6-70719 A

Non-Patent Document 1: Calcif. Tissue Int., 55: 230-235 (1994)

Non-Patent Document 2: Methods Enzymol., 107: 516-544 (1984)

### DISCLOSURE OF THE INVENTION

### Problems to be Solved by the Invention

As described above, the development of a method for effectively utilizing bones has been desired. In a conventional known method for extracting osteocalcin from a bone, raw bones are used as a raw material, which are extracted with a solvent containing EDTA or an acidic solvent. However, when used in food products, the application and the amount of EDTA is restricted; thus, the use of EDTA is inappropriate in the production of foodstuffs. Even if a step for removing EDTA is introduced to avoid this problem, complete removal of EDTA is difficult. Further, the introduction of such a step will increase the production cost. In the method using an acid, sufficient extraction efficiency cannot be obtained as compared with a method using EDTA. In addition, with respect to raw bones used as a raw material, their extracts have been already utilized as foodstuffs.

Therefore, an objective of the present invention is to efficiently extract active osteocalcin from a bone to provide an extract abundantly containing active osteocalcin suitable as a foodstuff.

### Means for Solving the Problem

The presence of useful proteins such as osteocalcin in an active form in heat-treated bones such as those extracted with hot water has not been revealed in the prior art. The present inventors have carried out a variety of extraction treatments of heat-treated bones by using food additives and making full use of food processing techniques. As a result, the present inventors have surprisingly found that active osteocalcin can be extracted at a high concentration with good efficiency, and that extracts excellent in safety and abundantly containing active osteocalcin can be prepared. The present inventors have also established a method for extracting active osteocalcin from heat-untreated raw bones, thereby completing the present invention to develop the use of active osteocalcin obtained from a bone.

The first aspect of the present invention relates to a method for producing an extract containing active osteocalcin, which comprises a step of extraction from a heat-treated bone with a solvent. In an embodiment of the first aspect of the present invention, the heat treatment can be moist heat treatment, and the solvent used for the extraction can be an acidic or alkaline aqueous solution. Examples of the heat-treated bones include boiled bones left after removing a soup made from pork bones.

The second aspect of the present invention relates to a method for producing an extract containing active osteocalcin, which comprises a step of solvent extraction from a heat-untreated bone with an alkaline aqueous solution.

In an embodiment of the first or second aspect of the present invention, an alkaline aqueous solution having a pH of 8 to 12 or an aqueous solution of carbonate and/or bicarbonate salts can be used as the alkaline aqueous solution. The method can also comprise a step of desalting the extract containing active osteocalcin.

The third aspect of the present invention relates to an extract containing active osteocalcin that is prepared using the method according to the first or second aspect of the present invention.

The fourth aspect of the present invention relates to a food or beverage product comprising the extract according to the third aspect of the present invention. Examples of the food or beverage product according to the fourth aspect of the present invention include food or beverage products for enhancing growth, for strengthening bones, and/or for osteoporosis. The container, package, and/or pamphlet for the food or beverage products according to the fourth aspect of the present invention can be accompanied by a label that indicates that the food or beverage product is used for enhancing growth, strengthening bones, and/or preventing or treating osteoporosis.

In addition, the present invention provides a food or beverage product comprising active osteocalcin that is used for enhancing growth, for strengthening bones, and/or for osteoporosis.

Furthermore, the present invention provides a method for producing a food or beverage product, which comprises a steps of: preparing an extract containing active osteocalcin by using the production method according to the first or second aspect of the present invention; and making indication that the food or beverage product is used for enhancing growth, strengthening bones, and/or preventing or treating osteoporosis.

Use of the extract according to the third aspect of the present invention for the production of a food or beverage product for enhancing growth, strengthening bones, and/or preventing or treating osteoporosis is also one of the embodiments of the present invention.

The present invention further provides a γ-carboxyglutamic acid (Gla) supplement containing the extract according to the third aspect of the present invention. Examples of the γ-carboxyglutamic acid (Gla) supplement include those provided in the form of a food or beverage product.

The fifth aspect of the present invention relates to a medicament comprising the extract according to the third aspect of the present invention.

The present invention also provides a medicament comprising active osteocalcin that is used for enhancing growth, strengthening bones, and/or preventing or treating osteoporosis.

The present invention further provides methods for enhancing growth, strengthening bones, and/or preventing or treating osteoporosis, which comprises a step of administering the extract according to the third aspect of the present invention.

Furthermore, the present invention provides methods for enhancing growth, strengthening bones, and/or preventing or treating osteoporosis, which comprises a step of administering active osteocalcin.

The sixth aspect of the present invention relates to an oral care composition comprising the extract according to the third aspect of the present invention.

The seventh aspect of the present invention relates to a feed comprising the extract according to the third aspect of the present invention.

The eighth aspect of the present invention relates to a growth enhancer comprising, as an active ingredient, active osteocalcin obtained from a bone.

The present invention also provides a cosmetic product comprising, as an active ingredient, the extract according to the third aspect of the present invention and/or active osteocalcin purified from the extract.

### EFFECTS OF THE INVENTION

According to the present invention, there is provided a method for producing an extract abundantly containing active osteocalcin. This production method allows the utilization of bone residues remaining after extraction of an extract on which useful applications have been desired. This production method also allows the preparation of an extract suitable for food or beverage products without using EDTA. Moreover, this method is more cost-saving than conventional production methods. The present invention also provides an extract containing active osteocalcin abundantly. The extract does not have any abnormal smell or taste and is a water-soluble extract from which fat components have been removed. It is suitable for food or beverage products applicable to various types of product forms. The present invention further provides a product comprising the extract such as a food or beverage product, a medicament, an oral care composition, or a feed.

### BEST MODE FOR CARRYING OUT THE INVENTION

In the present invention, bones used as a raw material are not particularly limited as long as they contain active osteocalcin. Specifically, bones of vertebrates, including meat animals such as cattle, pigs and chickens, and other mammals, birds and fishes can be used. Pork bones can be preferably used. Bones from which attached meat has been removed (hereinafter, sometimes, referred to as “raw bones”) can be used.

Surprisingly, the present inventors have found that an extract containing active osteocalcin can also be prepared from a bone treated with hot water or pressurized hot water. Thus, the present invention provides an extract containing active osteocalcin, which is obtained using a heat-treated bone as a raw material. A heat-treated bone has less extra fat components than a raw bone and is extremely preferred as a raw material for extracting active osteocalcin. In the present invention, examples of a heat-treated bone used as a raw material include that subjected to heat-treatment such as moist heat-treatment, for example hot water treatment, hot compressed water treatment, and/or high-pressure steam treatment. Examples of a bone subjected to such heat-treatment include a residue remaining after extraction of an extract from a bone (hereinafter, sometimes, referred to as a “bone extract-extraction residue”), for example, a residue remaining after extraction of an extract from a raw bone with steam (hereinafter, sometimes, referred to as a “bone steam extraction residue”) and a residue remaining after extraction of an extract from a raw bone with hot water (hereinafter, sometimes, referred to as a “bone hot water extraction residue”). Sometimes, a bone extract-extraction residue may be herein referred to as a boiled bone. When a heat-treated bone is used as a raw material in the present invention, the temperature employed for the heat-treatment, for example, moist heat treatment is not particularly limited, but the temperature can be, for example, 80 to 150° C., preferably 85 to 140° C., and more preferably 110 to 130° C. The heat-treatment time, for example, the moist heat treatment time is not particularly limited either, but a bone treated with heat for 15 minutes to 24 hours, preferably for 30 minutes to 10 hours, and more preferably for 1 to 8 hours can be used. In view of enhancing the extraction efficiency, a bone to be used as a raw material is preferably a crushed or pulverized raw or heat-treated (e.g., boiled) bone.

In the method of the present invention for producing an extract containing active osteocalcin from a heat-treated bone, an extraction solvent is not particularly limited, but an aqueous solvent can be preferably used as the extraction solvent. Examples thereof include various alkaline aqueous solutions, water, aqueous hydrochloric acid solution, aqueous acetic acid solution, aqueous phosphoric acid solution, aqueous lactic acid solution, aqueous citric acid solution, aqueous formic acid solution, and aqueous ascorbic acid solution. More preferably, an alkaline aqueous solution can be used. In the method for producing an extract containing active osteocalcin from a heat-untreated bone, use of an alkaline aqueous solution is preferred as described hereinafter. Use of food additives is preferred as raw materials for acidic and alkaline aqueous solvents.

Examples of the alkaline aqueous solution used in the present invention include aqueous solutions of carbonate and/or bicarbonate salts; aqueous solutions of boric acids, such as potassium borate and sodium borate; glycine-sodium hydroxide buffer; and ethanolamine acetate buffer.

Examples of the carbonate salt used in the present invention include sodium carbonate, potassium carbonate, magnesium carbonate, ammonium carbonate, calcium carbonate, and barium carbonate; and examples of the bicarbonate salt include sodium hydrogen carbonate, potassium hydrogen carbonate, and calcium hydrogen carbonate.

The pH of the preferred alkaline aqueous solution used in the present invention is pH>7 to 14, more preferably pH 8 to 13, and even more preferably pH 9 to 11. The salt concentration of the alkaline aqueous solution is preferably 0.001 M or higher, more preferably 0.1 to 3 M, and even more preferably 0.2 to 1 M. A method for adjusting pH of the alkaline aqueous solution of the present invention to a desired pH is not particularly limited, but the pH can be conveniently adjusted by incorporating, for example, both carbonate and bicarbonate salts at an appropriate ratio.

As another embodiment of the method for producing an extract containing active osteocalcin of the present invention, there is provided a method for producing an extract containing active osteocalcin, which comprises using a heat-untreated bone as a raw material, and an alkaline aqueous solution as an extraction solvent. The present inventors have found that active osteocalcin can be extracted from a heat-untreated bone in an efficient manner by using an alkaline aqueous solution as an extraction solvent without using EDTA. Examples of the alkaline aqueous solution used herein include those described above. The bone used as a raw material includes a variety of raw bones produced during meat processing.

In the production method of the present invention, the extraction time of active osteocalcin from a bone is not particularly limited, but the extraction time is preferably one hour or more, more preferably 6 to 48 hours, and even more preferably 12 to 24 hours. The extraction temperature is not particularly limited either, but the temperature is preferably 0 to 130° C., more preferably lower than 80° C., and even more preferably lower than 40° C.

When an alkaline aqueous solution is used as the extraction solvent, the production method of the present invention also includes a method for producing an extract containing active osteocalcin further comprising a step of neutralizing alkaline components contained in the extract obtained by the above production method with an acid. When the extract is used in a food or beverage product, the acid used for the neutralization is not particularly limited as long as it is acceptable to food processing, but examples of the acids to be used include inorganic acids such as hydrochloric acid, sulfuric acid, and nitric acid; organic acids such as adipic acid, citric acid, gluconic acid, succinic acid, formic acid, acetic acid, tartaric acid, lactic acid, malic acid and ascorbic acid; and solid acids such as a cation exchange resin, a cation exchange fiber and a cation exchange membrane. Among these, citric acid is the most preferred.

When an acidic aqueous solution is used as the extraction solvent, the production method of the present invention also includes a method for producing an extract containing active osteocalcin further comprising a step of neutralizing acidic components contained in the extract obtained in the above production method with an alkali component. When the extract is used in a food or beverage product, the alkali component used for the neutralization is not particularly limited as long as it is acceptable to food processing, but examples thereof include alkaline aqueous solutions containing an inorganic base (for example, an alkaline earth metal hydroxide; an alkaline metal carbonate such as sodium carbonate and potassium carbonate; an alkaline earth metal carbonate such as magnesium carbonate and barium carbonate; a alkaline metal hydrogen carbonate such as sodium hydrogen carbonate and potassium hydrogen carbonate; ammonium carbonate; and ammonium); alkaline aqueous solutions containing an organic base (for example, an organic acid salt of an alkaline metal such as sodium acetate and potassium propionate; an organic acid salt of an alkaline earth metal such as magnesium formate and magnesium acetate; amines; and nitrogen-containing heterocyclic compounds); and alkaline suspension containing an anion exchange resin. Among these, an aqueous sodium carbonate solution and a suspension of anion exchange resin can preferably be used.

The method for producing an extract of the present invention also includes a method for producing an extract containing active osteocalcin further comprising a step of desalting the extract obtained by the above production method. In the desalting step, complete removal of salts from the extract is not required. It is sufficient to carry out the desalting to such an extent that a salt concentration of the extract of the present invention becomes suitable for use in food or beverage products and the like. The desalting method used in the present invention is not particularly limited, and ultrafiltration, electrodialysis, gel filtration, and an ion-exchange resin can be used.

According to the method for producing an extract of the present invention, active osteocalcin can be extracted with high efficiency. The extraction efficiency of active osteocalcin in the production method of the present invention is not particularly limited because the efficiency varies depending on a particular shape of a bone used as a raw material and the content of active osteocalcin in the bone. For example, 80 μg or more (wet weight) of active osteocalcin can be obtained per 1 g of boiled pork bone. Active osteocalcin bound tightly to a bone is hardly expected to be absorbed into the body even if it is ingested. In contrast, active osteocalcin present in the extract obtained according to the production method of the present invention is water-soluble and capable of binding to minerals in a solution. Thus, the ingestion of the extract is expected to enhance the absorption of minerals including calcium.

As used herein, the term “extract” refers to a substance obtained by carrying out an extraction step using an extraction solvent. The extract of the present invention also includes substances obtained by further subjecting the above substance to treatments such as filtration, centrifugation, concentration, microfiltration, ultrafiltration, molecular sieving, and/or neutralization. The extract of the present invention also includes a fraction obtained by fractionating the above substance with a known method, or a fraction obtained by repeating the fractionation a plurality of times. Examples of the means for fractionation include extraction, fractional precipitation, column chromatography and the like. Furthermore, the extract of the present invention also includes a substance obtained by treating the above substance with an enzyme. Examples of the enzyme used for the enzyme treatment include proteases and peptidases such as pepsin, pancreatin, trypsin, papain, aminopeptidase and carboxypeptidase.

The extract of the present invention is characterized by a high content of active osteocalcin. According to the present invention, it is possible to provide, for example, an extract containing as dry weight 0.01% by weight or more of active osteocalcin, more specifically, an extract containing as dry weight 0.03 to 3.0% by weight of active osteocalcin. This extract makes it possible to prepare a product with a high concentration of active osteocalcin. The extract of the present invention may also contain inactive osteocalcin. It may also contain collagen and other proteins of bone origin.

In the present invention, the form of the extract of the present invention is not particularly limited and the extract can be any form of a powder, a solid and a liquid. To provide the extract in a powder form, any known method can be used, including, but not particularly limited to, spray-drying and freeze-drying. A powdered extract can be obtained, for example, by concentrating an extract obtained by extraction of active osteocalcin from a raw material with an extraction solvent, and then adding an excipient such as dextrin, sucrose fatty acid ester and lactose, followed by drying and grinding. A granulated solid obtained by granulating the extract according to a known method can also be used as the extract of the present invention. The granulation method is not particularly limited. Examples of the granulation method include tumbling granulation, agitation granulation, fluidized bed granulation, air-stream granulation, extruding granulation, compression molding granulation, disintegration granulation, jet granulation spray granulation, and spray-drying granulation. Examples of the liquid extract include a liquid obtained by the above-mentioned method for producing the extract itself, a concentrate or dilution thereof, as well as a solution prepared by dissolving the powdered extract described above into a liquid such as water and alcohol.

The food or beverage product of the present invention contains the extract described above. The food or beverage product of the present invention contains active osteocalcin abundantly, and is effective for strengthening the teeth and bones and useful for enhancing the absorption of minerals such as calcium. Minerals such as calcium can be incorporated into the food or beverage product. Examples of the food or beverage product include a physiologically functional food or beverage that is expected to have efficacy as described above. For example, such a functional food or beverage can also be provided as a health food (food or beverage for specified health use) that has a label indicating that it is used to obtain a desired effect produced by strengthening the teeth and bones or by enhancing the absorption of minerals such as calcium. The food or beverage product can also provides therapeutic and prophylactic effects on various diseases sensitive to the medicament of the present invention described hereinafter. When the extract contains collagen and/or other proteins of bone origin abundantly, it serves as a food or beverage product with a good cosmetic effect to the skin. Other components for strengthening the teeth and bones and/or for enhancing the absorption of minerals such as calcium can also be incorporated into the physiologically functional food or beverage. Examples of such components include, but not particularly limited to, vitamin D which is involved in absorption of calcium in the upper small intestine by active transport.

The method for producing the food or beverage product of the present invention is not particularly limited. For example, incorporation, cooking and processing of the extract of the present invention can be carried out by those employed in the production of conventional food or beverage products as long as the food or beverage product obtained contains the extract according to the present invention. The extract of the present invention itself can also be provided as a food or beverage.

As used herein for describing the food or beverage product of the present invention, the term “containing” refers to including, adding and/or diluting the extract of the present invention. In this context, the term “including” refers to an embodiment in which the food or beverage product includes the extract of the present invention therein; the term “adding” refers to an embodiment in which the extract of the present invention is added to the raw material of the food or beverage product; and the term “diluting” refers to an embodiment in which the raw material of the food or beverage product is added to the extract of the present invention.

The form of the food or beverage product of the present invention is not particularly limited as long as the product contains the aforementioned extract. The food or beverage product can be in the form suitable for oral ingestion including tablets, granules, capsules and the like. The aforementioned extract can also be prepared as a health food by adding glycerin and the like thereto. As used herein, the food or beverage product includes a beverage. The beverage of the present invention may be the extract of the present invention itself, or can be prepared by incorporating the extract into water or a known beverage. Further, examples of the food or beverage product of the present invention include dairy products such as milk and yogurt because they contain a high content of calcium.

The content of the extract of the present invention in the food or beverage product is not particularly limited. For example, the content of the extract of the present invention as dry weight is 0.01 to 100% by weight, more preferably 0.1 to 80% by weight, and even more preferably 0.5 to 50% by weight based on the food or beverage product.

Since the extract of the present invention contains a high content of active osteocalcin, a γ-carboxyglutamic acid (Gla) supplement containing the extract of the present invention is also provided as another embodiment of the present invention. The supplement is used to supply γ-carboxyglutamic acid into the body. A preferred embodiment of the supplement is a food or beverage product.

The medicament of the present invention contains the aforementioned extract. The medicament of the present invention contains active osteocalcin, and therefore it is useful for strengthening the teeth and bones (for example, increasing bone density or bone strength) and for enhancing the absorption of minerals such as calcium. A growth enhancing effect such as weight gain can also be obtained. Examples of diseases sensitive to the medicament of the present invention include osteoporosis (e.g. chronic osteoporosis, osteoporosis caused by postmenopausal hormone abnormalities, and idiopathic osteoporosis associated with diabetes and side-effects of drugs such as steroids), bone fracture, refracture, bone defect, dysosteogenesis, osteomalacia, Paget's disease of bone, rigid myelitis, chronic rheumatoid arthritis, osteoarthritis, osteoarthritis, growth disorders, periodontal disease, periodontal tissue destruction in periodontal disease, loss of the tooth root and socket.

Other components relating to strengthening the teeth and bones or enhancing the absorption of minerals such as calcium can also be incorporated into the medicament.

Usually, the medicament of the present invention can be prepared by incorporating the extract into a liquid or solid pharmaceutically acceptable carrier and, if desired, by adding other agents such as solvents, dispersants, emulsifiers, buffers, stabilizers, excipients, binders, disintegrants and lubricants to obtain a solid formulation such as tablet, granule, particle, powder and capsule, or a liquid formulation such as solution, suspension and emulsion. The medicament can also be prepared as dry product that can be formulated into a liquid formulation by addition of an appropriate carrier before use, as well as formulated into an external formulation.

The pharmaceutical carrier can be appropriately selected according to a particular administration route and a dosage form of the medicament of the present invention. For preparing an oral formulation comprising a solid composition, the formulation can be in the form of tablets, pills, capsules, powders, fine granules, granules or the like. Examples of the carrier include starch, lactose, sucrose, mannitol, carboxymethyl cellulose, corn starch, inorganic salts and the like. For preparing an oral formulation, other agents can also be incoporated thereinto. Examples thereof include binders, disintegrants, surfactants, lubricants, fluidity accelerators, flavoring agents, colorants, and spices. For example, for preparing tablets or pills, they can be covered, if desired, with a sugar-coating of sucrose, gelatin or hydroxypropyl cellulose, or with a film of a gastric or enteric material. For preparing an oral formulation comprising a liquid composition, the formulation can be in the form of a pharmaceutically acceptable emulsion, solution, suspension, syrup, or the like. Examples of the carrier thereof include purified water, ethanol or the like. Further, if desired, other agents can also be added. Examples thereof include auxiliary agents such as wetting agents and suspending agents, sweeteners, flavoring agents, and antiseptics. Since the extract of the present invention also shows a sufficient effect by oral administration, the medicament for oral administration is one of preferred embodiment of the present invention. This is also preferred in view of convenience of administration.

On the other hand, for preparing a parenteral formulation, it can be prepared by dissolving or suspending the extract of the present invention in a diluent such as distilled water for injection, physiological saline, an aqueous solution of glucose, vegetable oil for injection, sesame oil, peanut oil, soybean oil, corn oil, propylene glycol or polyethylene glycol according to a conventional method. If necessary, other agents can be added. Examples thereof include disinfectants, stabilizers, tonicity agents, and soothing agents. It is also possible to prepare a solid composition, which is dissolved in sterile water or a sterile solvent for injection before use.

The external formulation include solid, semi-solid or liquid formulations for percutaneous administration or transmucosal (oral or intranasal) administration. The external formulation also includes suppositories or the like. The external formulation can be in the form of, for example, emulsions such as milky liquids and lotions; liquid formulations such as external tinctures and liquid preparations for transmucosal administration; ointments such as oily ointments and hydrophilic ointments; patches for percutaneous or transmucosal administration such as films, tapes and poultices.

The various formulations described above can be appropriately prepared according to conventional methods using known pharmaceutical carriers and the like. The content of the extract in these formulations is not particularly limited as long as it is in an amount that can preferably be administered in the dose range described below by taking the dosage forms, the administration method and the like into the consideration. The content of the extract of the present invention contained in these formulations can be about 0.1 to 100% by weight as the dry weight of the extract.

The dose of the medicament of the present invention varies and is appropriately determined according to a particular dosage form, administration method, intended use, and age, body weight, and conditions of the subject to be administered. In general, the daily dose for an adult as expressed by the dry weight of the extract contained in the formulation for example, is 0.1 μg to 10 g/kg body weight, preferably 1 μg to 1 g/kg body weight, and more preferably 10 μg to 0.1 g/kg body weight. As a matter of course, since the dose varied depending on various conditions, an amount less than the above-described dose may be sufficient, or an amount exceeding the range may be required. The administration may be carried out with a single dose or with a number of doses in a day within the desired dose range. Any dosing period may be employed. The medicament of the present invention can be administered directly in the mouth, or can be taken, on a daily basis, as any food or beverage product to which the medicament is added.

The oral care composition of the present invention is characterized by containing the extract described above. The oral care composition of the present invention is expected to promote calcification of the teeth since it contains active osteocalcin. The oral care composition of the present invention can be in the form of liquid, paste, ointment and powder. The oral care composition of the present invention can be coated on or incorporated into a fiber carrier such as floss. The composition can be in the form of toothpaste, dental rinse, gum, mouthwash, mouth spray, toothbrush, dental floss, or interdental cleaner. The content of the extract of the present invention contained in the oral care composition is not particular limited. For example, the content can be 0.1 to 100% by weight, preferably 0.5 to 80% by weight, and more preferably 1 to 50% by weight as the dry weight of the extract of the present invention in the oral care composition.

The feed of the present invention is characterized by containing the extract described above. Since the feed of the present invention contains active osteocalcin, the growth and bone strength of organisms such as livestock animals, experimental animals, poultry, fish and pet animals can be enhanced by feeding them with the feed of the present invention. Furthermore, the feed of the present invention is expected to show the same effect as that provided by the medicament of the present invention as described above based on the physiological effect of osteocalcin used in the present invention.

The organisms fed with the feed of the present invention are not particularly limited. Examples thereof include livestock animals such as horses, cattle, pigs, sheep, goats, camels and llama; experimental animals such as mice, rats, guinea pigs and rabbits; poultry such as chickens, ducks, turkeys and ostriches; fishes such as red sea breams, Japanese parrot fishes, Japanese flounders, flounders, Japanese amberjacks, yellow tails, goldstriped amberjacks, tunas, Japanese horse mackerels, Japanese trouts, salmons and trouts, tiger puffer fishes, eels, oriental weatherfishes, and catfishes; crustaceans such as Japanese tiger prawns, black tiger prawns, fleshy prawns and swimming crabs; shellfishes such as abalones, turban shells, scallops and oysters; and pet animals such as dogs and cats.

In general, the feed used in the present invention is fed, as expressed by the dry weight of the extract, at a feed dose of 0.0001 to 2000 mg/kg body weight, preferably 0.001 to 1000 mg/kg body weight, and more preferably 0.01 to 100 mg/kg body weight of the subject organism per day. As a matter of course, the feed dose can vary depending on various conditions. An amount less than the above-described feed dose may be sufficient, or an amount exceeding the range may be required. The feeding can be carried out, for example, by adding/mixing the extract of the present invention to/with a raw material for an artificial mixed feed, or by mixing the extract with a powdered raw material of an artificial mixed feed and then further adding/mixing the extract to other raw materials.

Examples of the raw materials for the artificial mixed feed described above include animal-derived materials such as fish flour, casein, meat meal and squid meal; plant-derived materials such as soy meal, wheat flour, starch, corn gluten meal and feed yeasts; animal oils such as cod liver oil and squid liver oil; vegetable oils such as soy oil and rapeseed oil; vitamins; minerals; amino acids; and antioxidants. The feed of the present invention can be provided by mixing the active ingredient of the present invention with the artificial mixed feed prepared by mixing raw materials as described above.

The content of the extract of the present invention contained in the feed is not particularly limited. The content can be 0.0001% or more by weight, preferably 0.001 to 100% by weight, and more preferably 0.01 to 100% by weight based on 100% by weight of the feed.

For preparing an artificial mixed feed for fish, in addition to the extract of the present invention, a composition having an antiviral effect can be mixed with the artificial feed in view of preventing infection of fish with pathogenic microorganisms. Examples of the antiviral composition include those including a cyclopentenone derivative such as processed kelp products rich in DHCP (4,5-dihydroxy-2-cyclopentan-1-one).

The feed of the present invention can be fed to the subject organisms as a beverage. Where the feed is fed as a beverage, the concentration of the active ingredient of the present invention is not particularly limited. The concentration varies depending on the intended use, but 0.0001 to 1 w/w % is suitable.

The growth enhancer of the present invention is characterized by containing, as an active ingredient, active osteocalcin obtained from a bone. The growth enhancer of the present invention has an enhancing effect on the growth of animals such as human beings, livestock animals and cultivated fish. For example, the increase in body height and weight of animals is expected as the effect of the growth enhancer of the present invention. The growth enhancer can be produced according to a conventional method as long as the active ingredient described above is contained therein. Food products containing the growth enhancer are included in this embodiment of the present invention. Such food products can be used as “foods with a growth-enhancing effect” or “foods for enhancing growth” with a label indicating, for example, the growth-enhancing effect or its use for enhancing the growth.

No lethal case is observed in rats administered with a single oral dose of 200 mg/kg of active osteocalcin obtained according to the present invention.

The present invention provides an efficient method for extracting active osteocalcin. It also provides safe additives for medicaments and food or beverage products that contain the extract as their active ingredients.

The extract of the present invention contains, in addition to active osteocalcin, a group of proteins including collagen originated from the bones used as a raw material as a main component, abundantly, and it can be produced on a large scale and marketed as health foods for maintaining the health of bones and for increasing beauty, and as a material for physiologically functional food products.

For example, extracts have been produced from pork bones from which the meat has been removed, and commercialized as ramen broth and the like. Kagoshima prefecture is a representative stock-raising prefecture in Japan, wherein approximately 70,000 tons of pork bones are produced in a year, and bone extract-extraction residues, i.e., boiled bones are also produced in large amounts. However, active osteocalcin has not been utilized heretofore as a material for physiologically functional food products since it is bound tightly to bones. The present inventors have extensively investigated extraction of active osteocalcin, a protein essential for osteogenesis. As a result, it has been possible to prepare, for example, 700 g of an extract containing 2.1 g of active osteocalcin from 30 kg of boiled pork bones, using the food-processing techniques possessed by the present inventors.

The present invention has made it possible for the first time to provide a safe extract rich in active osteocalcin. Osteocalcin is a protein produced by osteoblasts and capable of accumulating in a bone only in its active form. The extract according to the present invention contains such an active form of osteocalcin, and it is useful for altering the balance between bone destruction and formation in favor of formation. As an embodiment of the present invention, a water-soluble extract is provided which has no abnormal smell or taste and is free from fat components. This type of extract rich in active osteocalcin can be applied to a variety of product forms.

### EXAMPLES

Hereinafter, the present invention will be illustrated in detail with reference to Examples, but is not limited thereto.

### Example 1

### Investigation of Solvents for Extraction of Active Osteocalcin from Pork Bones (1)

The extraction solvents were prepared according to the following procedure. 0.2 M Carbonate buffer (pH 9.5): 0.275 g of sodium carbonate (NACALAI TESQUE, INC.) and 0.62 g of sodium hydrogen carbonate (NACALAI TESQUE, INC.) were dissolved in distilled water to make up to 50 mL. 1 M Hydrochloric acid: 45.7 mL of distilled water was mixed with 4.3 mL of hydrochloric acid (NACALAI TESQUE, INC.). 1 M Phosphate buffer (pH 6.7): 7.1 g of disodium hydrogenphosphate (NACALAI TESQUE, INC.) dissolved in distilled water to make up to 50 mL and 6.8 g of monopotassium dihydrogen phosphate (NACALAI TESQUE, INC.) dissolved in distilled water to make up to 50 mL, followed by mixing both solutions and adjusting to pH 6.7. Then, 10 mL of each extraction solvent was added to 3 g (wet weight) of mixer-ground bone extract extraction residues (boiled bones) of raw pork bones with hot compressed water, and the mixture was extracted with stirring for 24 hours at 4° C. After separation of the extract from residues, the extract was neutralized and then desalted by dialysis (SPECTRUM LABORATORIES, INC.; molecular-weight cutoff 3500). Subsequently, the content of active osteocalcin in the dialyzed solution was determined by ELISA (TAKARA BIO INC.). The results are shown in Table 1. As shown in Table 1, it was demonstrated that the carbonate buffer had particularly high extraction efficiency for active osteocalcin.

TABLE 1

| | Amount of active osteocalcin |
|---|---|
| | extracted from 1 g of raw |
| Extraction solvent | material (μg) |
| 0.2 M carbonate buffer (pH 9.5) | 292.2 |
| 1 M hydrochloric acid | 76.8 |
| 1 M phosphate buffer (pH 6.7) | 57.3 |

### Example 2

### Investigation of Raw Materials for Extraction of Active Osteocalcin from Pork Bones

Raw pork bones were heated in how water (90° C.) for 30 minutes to obtain heated-extract solution 1. The residual pork bones were taken out and coarsely ground. Then, the pork bones coarsely ground were heated again in how water (90° C.) for 30 minutes to obtain heated-extract solution 2 and pork bones treated with hot water. Subsequently, raw pork bones were heated in compressed hot water (120° C.) for five hours to obtain heated-extract solution 3 and pork bones treated with compressed hot water. Then, according to the same manner as that in Example 1, extracts were prepared and the content of active osteocalcin in the extracts was determined except that 20 g (wet weight) of the pork bones treated with hot water or 20 g (wet weight) of the pork bones treated with compressed hot water, both of which were used after coarsely grinding with a mixer, were used as the raw material bones, and that 60 mL of 0.5 M carbonate buffer (pH 9.5) was used as the extraction solvent. The results are shown in Table 2. As shown in Table 2, it was demonstrated that active osteocalcin was extracted into the carbonate buffer from the pork bones treated with hot water and the pork bones treated with compressed-hot water. Active osteocalcin was scarcely detected in heated-extract solutions 1 to 3.

TABLE 2

| | Amount of active osteocalcin |
|---|---|
| | extracted from 1 g of raw |
| Raw material | material (μg) |
| pork bones treated with hot | 33.9 |
| water | |
| pork bones treated with | 90.9 |
| compressed hot water | |

### Example 3

### Investigation of Salt Concentrations of Extraction Solvent for Extraction of Active Osteocalcin from Pork Bones

Various concentrations of carbonate buffer were prepared as follows. 1 M Carbonate buffer (pH 9.5): 1.375 g of sodium carbonate and 3.1 g of sodium hydrogen carbonate were dissolved in distilled water to make up to 50 mL. This solution was diluted with distilled water to give 0.05 to 0.5 M carbonate buffers (pH 9.5). Then, according to the same method as that in Example 1, the correlation between the concentration of carbonate buffers (pH 9.5) and the extraction efficiency of active osteocalcin was investigated. The results are shown in Table 3. As shown in Table 3, it was demonstrated that while active osteocalcin was extracted at all the concentrations tested, the extraction efficiency of active osteocalcin was particularly high in 0.1 to 1 M carbonate buffers (pH 9.5).

TABLE 3

| | Amount of active osteocalcin extracted |
|---|---|
| Concentration (M) | from 1 g of raw material (μg) |
| 0.05 | 58.0 |
| 0.1 | 147.2 |
| 0.2 | 205.2 |
| 0.5 | 410.2 |
| 1.0 | 395.3 |

### Example 4

### Investigation of Extraction Temperatures for Extraction of Active Osteocalcin from Pork Bones

According to the same method as that in Example 1, the extraction temperatures for extracting active osteocalcin in 0.2 M carbonate buffer (pH 9.5) were investigated except that 0.2 M carbonate buffer (pH 9.5) was used as the extraction solvent at the extraction temperatures of 4° C. and 30° C. The results are shown in Table 4. As shown in Table 4, it was demonstrated that the extraction efficiency of active osteocalcin was higher at 30° C.

TABLE 4

| | Amount of active osteocalcin extracted from |
|---|---|
| Temperature (° C.) | 1 g of raw material (μg) |
| 4 | 205.2 |
| 30 | 351.7 |

### Example 5

### Investigation of pH of Extraction Solvent for Extraction of Active Osteocalcin from Pork Bones

Carbonate and bicarbonate solutions were prepared as follows. A 0.5 M bicarbonate solution (pH 8.0): 2.1 g of sodium hydrogen carbonate was dissolved in distilled water to make up to 50 mL. A 0.5 M carbonate solution (pH 12.0): 2.65 g of sodium carbonate was dissolved in distilled water to make up to 50 mL. Then, the pH of the extraction solvents for extraction of active osteocalcin was investigated using the 0.5 M bicarbonate solution and 0.5 M carbonate solution. The results are shown in Table 5. As shown in Table 5, it was demonstrated that active osteocalcin was extracted with high efficiency using a carbonate and/or dicarbonate solution at pH ranging from 8 to 12.

TABLE 5

| | Amount of active osteocalcin extracted from 1 g |
|---|---|
| pH | of raw material (μg) |
| 8.0 | 164.0 |
| 12.0 | 228.5 |

### Example 6

### Investigation of Solvents for Extraction of Active Osteocalcin from Pork Bones (2)

According to the same method as that in Example 2, extracts were prepared from the mixer-ground pork bones treated with compressed hot water except that distilled water, 0.5 M acetate, 26.2% lactate, 0.25 M and 0.5 M glycine-sodium hydroxide buffer (pH 9.5), 0.5 M bicarbonate solution (pH 8.0), 0.5 M carbonate buffer (pH 9.5), and 0.5 M sodium carbonate solution (pH 12.0) were used as the extraction solvents to evaluate the extraction efficiency of active osteocalcin using various solvents. The results are shown in Table 6. As shown in Table 6, it was demonstrated that 0.25 M and 0.5 M glycine-sodium hydroxide buffer (pH 9.5), 0.5 M bicarbonate solution (pH 8.0), 0.5 M carbonate buffer (pH 9.5), and 0.5 M sodium carbonate solution (pH 12.0) showed higher extraction efficiency for active osteocalcin, as compared with distilled water, 0.5 M acetate, and 26.2% lactate.

TABLE 6

| | Amount of active |
|---|---|
| | osteocalcin extracted |
| | from 1 g of raw |
| Extraction solvent | material (μg) |
| distilled water | 9.1 |
| 0.5 M acetate | 6.3 |
| 26.2% lactate | 23.7 |
| 0.25 M glycine-sodium hydroxide buffer | 90.9 |
| (pH 9.5) | |
| 0.5 M glycine-sodium hydroxide buffer | 83.3 |
| (pH 9.5) | |
| 0.5 M bicarbonate solution | 48.2 |
| (pH 8.0) | |
| 0.5 M carbonate buffer (pH 9.5) | 92.7 |
| 0.5 M sodium carbonate solution | 67.3 |
| (pH 12.0) | |

### Example 7

### Investigation of Solvents for Extraction of Active Osteocalcin from Pork Bones (3)

According to the same method as that described in Example 6, extraction efficiency of active osteocalcin was evaluated except that 1 M hydrochloride solution, 0.5 M glycine-sodium hydroxide buffer (pH 9.5 and 10.5), 0.5 M carbonate buffer (pH 9.5), 0.5 M potassium borate buffer (pH 9.1), 0.5 M sodium borate buffer (pH 9.1), and 0.5 M ethanolamine-acetate buffer (pH 9.6) were used as the extraction solvents. The results are shown in Table 7. As shown in Table 7, it was demonstrated that 0.5 M glycine-sodium hydroxide buffer (pH 9.5 and 10.5), 0.5 M carbonate buffer (pH 9.5), 0.5 M potassium borate buffer (pH 9.1), 0.5 M sodium borate buffer (pH 9.1), and 0.5 M ethanolamine acetate buffer (pH 9.6) showed higher extraction efficiency for active osteocalcin as compared with 1 M hydrochloride solution.

TABLE 7

| | Amount of active |
|---|---|
| | osteocalcin extracted |
| | from 1 g of raw |
| Extraction solvent | material (μg) |
| 1 M hydrochloride solution | 15.7 |
| 0.5 M glycine-sodium hydroxide buffer | 77.9 |
| (pH 9.5) | |
| 0.5 M glycine-sodium hydroxide buffer | 104.2 |
| (pH 10.5) | |
| 0.5 M carbonate buffer (pH 9.5) | 149.8 |
| 0.5 M potassium borate buffer (pH 9.1) | 127.1 |
| 0.5 M sodium borate buffer (pH 9.1) | 84.0 |
| 0.5 M ethanolamine acetate buffer | 109.8 |
| (pH 9.6) | |

### Example 8

To 30 kg (wet weight) of residual bones (boiled bones) ground with a mixer after extraction of the raw pork bones with compressed hot water was added 150 L of 0.5 M carbonate buffer (pH 9.5), and the extraction was carried out with stirring for 22 hours at room temperature. The extract was separated from the residues and then subjected sequentially to ceramic membrane filtration, diatomite filtration, aseptic filtration, ultrafiltration, and aseptic filtration to obtain 36 L of the sterile filtrate. The filtrate was then lyophilized to prepare 410 g of a lyophilized material containing 1.7 g of active osteocalcin.

### Example 9

According to a conventional method, green tea was prepared by using 10 g of green tea leaves, 0.2 g of vitamin C and 1000 mL of ion-exchanged water. The product 1 of the present invention was prepared by adding the lyophilized material prepared as described in Example 8 so that 30 mg of active osteocalcin was contained in 100 mL of the product 1. The green tea without the additive was used as a control. The flavor of the product 1 of the present invention was similar to the control, and no animal odor or the like was recognized.

### Example 10

The product 2 of the present invention was prepared by using homogenized regular milk (88.6 w/v % water, 2.8 w/v % protein, 3.5 w/v % fat, 4.5 w/v % lactose, and 0.8 w/v % minerals) with addition of the lyophilized material prepared as described in Example 8 so that 30 mg of active osteocalcin was contained in 100 mL of the product 2. The milk without the additive was used as a control. The flavor of the product 2 of the present invention was similar to the control and no increase in animal odor was recognized.

### Example 11

According to a conventional method, soy milk was prepared from soy beans and solidified with a solidifying agent to prepare regular firm tofu. The product 3 of the present invention was prepared by adding the lyophilized material prepared as described in Example 8 so that 40 mg of active osteocalcin was contained in 100 mL of the product 3. The tofu without the additive was used as a control. The flavor of the product 3 of the present invention was similar to the control, and no animal odor or the like was recognized.

### Example 12

As sweets, orange jelly was produced in a small scale. The orange jelly was prepared by mixing 9 g of carrageenan with 180 g of granulated sugar, adding 800 mL of water thereto, followed by melting the mixture by heating with stirring. To the melted mixture were added 10 g of a mandarin orange juice concentrate, 2 g of citric acid, 1.5 g of sodium citrate, 2 g of orange aroma, and 1 g of flavor. The product 4 of the present invention was prepared by adding the lyophilized material as described in Example 8 so that 20 mg of active osteocalcin was contained in 100 g of the product 4. The orange jelly without the additive was used as a control. The flavor of the product 4 of the present invention was similar to the control, and no animal odor or the like was recognized.

### Example 13

As meat paste products, sausages were produced in a small scale. The sausages were prepared by mincing 2 kg of pork meat and 700 g of pork fat with a 5 mm plate mincer and mixing with 7 g of pepper, 3 g of sage, and 1 g of mace. The mixture was cut and stuffed into a hog casing with a diameter of 2 cm, followed by steaming for 15 minutes to obtain the sausages. Before cutting the mixture, in the product 5 of the present invention, the lyophilized material prepared as described in Example 8 was added so that 50 mg of active osteocalcin was contained in 100 g of the product 4. The sausages without the additive were used as a control. The flavor of the product 5 of the present invention was similar to the control, and no increase in animal odor was recognized.

### Example 14

According to a conventional method, tablets containing the lyophilized material prepared as described in Example 8 (200 mg/tablet) were prepared at the mixing ratio shown in Table 8 hereinafter using a tableting machine at a tableting pressure of 3000 kg/cm².

TABLE 8

| Component | mg/tablet |
|---|---|
| Lyophilized material containing active | 25 |
| osteocalcin | |
| Crystalline cellulose | 96 |
| Starch | 54 |
| Sucrose fatty acid ester | 5 |
| Calcium carbonate | 20 |
| Total | 200 |

### Example 15

A toothpaste composition containing the lyophilized material prepared as described in Example 8 was prepared at the mixing ratio shown in the following Table 9.

TABLE 9

| Component | % by weight |
|---|---|
| Calcium hydrogen phosphate dihydrate | 40 |
| Calcium hydrogen phosphate anhydrate | 2 |
| Glycerin | 7.25 |
| Solbit | 7.25 |
| Carboxymethyl cellulose | 1 |
| Sodium lauryl sulfate | 2 |
| Saccharine sodium | 0.5 |
| Flavoring agent | 1 |
| Lyophilized material containing active | 10 |
| osteocalcin | |
| Purified water | balance |
| Total | 100 |

### Example 16

A chewing gum composition containing the lyophilized material prepared as described in Example 8 was prepared at the mixing ratio shown in the following Table 10.

TABLE 10

| Component | % by weight |
|---|---|
| Gum base | 64 |
| Xylitol | 25 |
| Flavoring agent | 1 |
| Lyophilized material containing active | 10 |
| osteocalcin | |
| Total | 100 |

### Example 17

To 30 kg (wet weight) of residual bones (boiled bones) ground with a mixer after extraction of the raw pork bones with compressed hot water was added 90 L of 0.5 M carbonate buffer (pH 9.5), and the extraction was carried out with stirring for 24 hours at room temperature. The extract obtained by separating from the residues was neutralized with 0.5 M citrate aqueous solution. Subsequently, the extract was subjected to ultrafiltration to obtain 13.3 L of the filtrate. The filtrate was then lyophilized to prepare 700 g of a lyophilized material containing 2.1 g of active osteocalcin.

### Example 18

Fifty chickens (female) of 6-week post-hatch were fed with a feed containing 0.5% (w/w) of the lyophilized material prepared in Example 17. A group of 100 chickens were fed with a feed without the lyophilized material as a control. Nine weeks after the feeding, the chickens were weighed and measured. Chickens of the control group weighed 1185.3±114 g and chickens of the lyophilized material-treated group weighed 1260.2±101.7 g, showing that the extract of the present invention had a significant enhancing effect on the growth of chickens.

### Example 19

To 600 kg (wet weight) of residual bones (boiled bones) ground with a mixer after extraction of the raw pork bones with compressed hot water was added 3000 L of 0.5 M carbonate buffer (pH 9.5), and the extraction was carried out with stirring for 22 hours at room temperature. The extract was separated from the residues and then subjected sequentially to ceramic membrane filtration, diatomite filtration, aseptic filtration, ultrafiltration, and aseptic filtration, thereby obtaining 700 L of the sterile filtrate. The sterile filtrate thus obtained was then concentrated with an evaporator to a volume of 85 L. To this concentrate, dextrin (Matsutani Chemical Industry Co., Ltd.: Pinedex #1) was added at a proportion of 80% by solid weight of the concentrate, followed by spray-drying to prepare a composition containing osteocalcin for blending feeds.

### Example 20

An enzyme-treated extract containing osteocalcin was prepared as follows. Ten g of the lyophilized material prepared as described in Example 8 was dissolved in 50 mL of ion-exchanged water, and 0.25 g of pancreatin (SIGMA-ALDRICH Japan K.K.) was added at pH 6.8 to allow the enzymatic reaction at 37° C. for 1 hour, thereby obtaining the enzyme-treated extract containing osteocalcin. To obtain a control-treated product, the same reaction was carried out without the lyophilized material prepared as described in Example 8.

Subsequently, the enzyme-treated extract containing osteocalcin was evaluated for the improving effect on the intestinal absorption of calcium.

Nine-week old male Wister rats (n=5) were fasted overnight, and then small intestine was excised and divided into segments of 3 cm each, from the junction between ileum and caecum (ileocecal junction) toward duodenum. Thus, six to eight specimens were obtained from each rat. Each specimen was carefully reversed and stitched at both end portions to prepare a reversed intestinal sack.

Then, into each reversed intestinal sack, 0.3 mL of chorionic membrane buffer (50 mM Tris, 150 mM NaCl, 4 mM KCl, 10 mM D-glucose, and 1.25 mM CaCl₂ dihydrate, pH 7.6) was dispensed. The enzyme-treated extract containing osteocalcin warmed at 37° C. or the control-treated product was incubated in a 20-fold diluted mucosal membrane buffer (50 mM Tris, 150 mM NaCl, 4 mM KCl, 10 mM D-glucose, and 10 mM CaCl₂ dihydrate, pH 7.6). After incubated for 15 minutes, the fluid in the sack was sampled from the cannula inserted into the sack and the calcium concentration in the fluid was determined using the Calcium E test WAKO kit (Wako Pure Chemical Industries, Ltd.) to calculate calcium absorption. The results are shown in Table 11.

TABLE 11

| | Calcium absorption |
|---|---|
| ^{▪} | (μg/cm sack)^{▪} |
| Control-treated group^{▪} | 5.9 ± 0.36^{▪} |
| Enzyme-treated extract group | 7.4 ± 0.81^{▪} |
| containing osteocalcin^{▪} | |
| ^{▪} | Mean ± standard error^{▪} |

As shown in Table 11, it was demonstrated that the Enzyme-treated extract containing osteocalcin enhanced calcium absorption in the reversed intestinal sack model.

### Example 21

Four-week old male Wister rats were habituated, and then divided into the groups of rats administered extracts containing osteocalcin (n=5) and control rats (n=5). The rats of the group administered extracts containing osteocalcin were fed with a standard meal (CE-2; CLEA Japan, Inc.) containing 2.5% by weight of the lyophilized material prepared as described in Example 8. The rats of the control group were fed with the standard meal. At day 10 after administration, blood was collected from the rats of each group, and the blood calcium concentration was determined using the Calcium E test WAKO kit. The results are shown in Table 12.

TABLE 12

| | Blood calcium |
|---|---|
| ^{▪} | concentration (mg/dL)^{▪} |
| Control group^{▪} | 10.0 ± 0.07^{▪} |
| Group administered extracts | 10.4 ± 0.06^{▪} |
| containing osteocalcin^{▪} | |
| ^{▪} | Mean ± standard error^{▪} |

As shown in Table 12, it was demonstrated that the blood calcium concentration was higher in the group administered extracts containing osteocalcin as compared with the control group.

### Example 22

Eight-week old female Wister rats were divided into the oophorectomy group (n=13) and the mock surgery group (n=6), and subjected to oophorectomy and mock surgery, respectively. The rats of the oophorectomy group were further divided into the group administered extracts containing osteocalcin (n=7), in which the rats habituated for two weeks with a low-calcium feed (0.1% calcium content; CLEA Japan, Inc.) and then fed with a low-calcium feed (0.1% calcium content) containing 2.5% by weight of the lyophilized material prepared as described in Example 8, and the control group (n=6), in which the rats were fed with the same low-calcium feed for 8 weeks after the habituation. The rats of the mock surgery group were fed a low-calcium feed for 10 weeks after the mock surgery. Blood was collected from the rats of each group at week 10 after surgery.

Subsequently, the rats were bled to death under anesthesia and evaluated for bone density of the metaphysis by isolating the femur. Bone density was measured at 3 mm from the distal growth plate by the pQCT method (Practical Guide for Mechanical Testing of Bone, CRC Press, pp. 385-405, 1999). The results are shown in Table 13.

TABLE 13

| | Total bone density |
|---|---|
| | (mg/cm³) |
| Control group | 459.7 ± 3.9 |
| Group administered extracts | 471.2 ± 4.4 |
| containing osteocalcin | |
| Mock surgery group | 532.6 ± 6.5 |

Mean ± standard error

As shown in Table 13, it was demonstrated that the extract containing osteocalcin has an increasing effect on the bone density of the metaphysic of femur.

The calcium concentration in the blood collected at week 10 after surgery was determined. The results are shown in Table 14.

TABLE 14

| | Blood calcium concentration^{▪} |
|---|---|
| ^{▪} | (mg/dL)^{▪} |
| Control group | 9.2 ± 0.15^{▪} |
| Group administered | 9.7 ± 0.15^{▪} |
| extracts containing | |
| osteocalcin | |
| Mock surgery group | 10.2 ± 0.22^{▪} |
| ^{▪} | Mean ± standard error^{▪} |

As shown in Table 14, it was demonstrated that the blood calcium concentration was higher in the osteocalcin-containing extract-administered group as compared with the control group.

### Example 23

Four-week old male Wister rats were habituated with a low-calcium feed (0.1% calcium content), and then divided into the group administered extracts containing osteocalcin (n=5) and the control group (n=5). The rats of the group administered extracts containing osteocalcin were fed with a low-calcium feed (0.1% calcium content) containing 2.5% by weight of the lyophilized material prepared as described in Example 8. On the other hand, the rats of the control group were fed with a low-calcium feed (0.1% calcium content). At day 10 after administration, the rats were bled to death under anesthesia and evaluated for bone density of the metaphysis by isolating the femur. Bone density was measured at the portion of 12 mm from the distal growth plate by the pQCT method. The results are shown in Table 15.

TABLE 15

| | | | Three- | |
|---|---|---|---|---|
| | Total | Cortical | point | |
| | bone | bone | bending | Cortical bone |
| | density^{▪} | density^{▪} | strength^{▪} | thickness^{▪} |
| ^{▪} | (mg/cm³)^{▪} | (mg/cm³)^{▪} | (mm³)^{▪} | (mm)^{▪} |
| Control | 485.4^{▪} ± 10.8^{▪} | 1037.3^{▪} ± 6.7^{▪} | 1.428^{▪} ± 0.048^{▪} | 0.300^{▪} ± 0.008^{▪} |
| group | | | | |
| Group | 497.8^{▪} ± 19.9^{▪} | 1041.8^{▪} ± 16.2^{▪} | 1.489^{▪} ± 0.033^{▪} | 0.315^{▪} ± 0.010^{▪} |
| administered | | | | |
| extracts | | | | |
| containing | | | | |
| osteocalcin | | | | |
| ^{▪} | ^{▪} | ^{▪} | ^{▪} | Mean ± |
| | | | | standard |
| | | | | error^{▪} |

As shown in Table 15, it was demonstrated that the group administered extracts containing osteocalcin showed higher bone density in the femoral diaphysis as compared with the control group, indicating that the cortical bone thickness and the three-point bending strength were increased.

### INDUSTRIAL APPLICABILITY

According to the present invention, an extract containing active osteocalcin suitable for incorporating in medicaments, and food and beverage products can be produced at a low cost. Further, a product having a high added value can be produced from food-processing wastes.

1. A process for producing an extract containing active osteocalcin, which comprises a step of solvent extraction from a bone subjected to heat treatment.

2. The process according to claim 1, wherein the heat treatment is moist heat treatment.

3. The process according to claim 1, wherein a solvent used for the extraction is an acidic aqueous solution or an alkaline aqueous solution.

4. A process for producing an extract containing active osteocalcin, which comprises a step of solvent extraction from a bone without heat treatment with an alkaline aqueous solution.

5. The process according to claim 3, wherein the alkaline aqueous solution has a pH 8 to 12.

6. The process according to claim 3, wherein the alkaline aqueous solution is an aqueous solution of a carbonate salt and/or a bicarbonate salt.

7. The process according to claim 1, which further comprises a step of desalting the extract containing active osteocalcin.

8. An extract containing active osteocalcin, which is prepared by the process according to claim 1.

9. A food or beverage product comprising the extract according to claim 8.

10. A medicament comprising the extract according to claim 8.

11. An oral care composition comprising the extract according to claim 8.

12. A feed comprising the extract according to claim 8.

13. A growth enhancer comprising as an active ingredient active osteocalcin obtained from a bone.
